(19) United States
(12) Patent Application Publication
(10) Pub. No.: US 2003/0224045 A1
(43) Pub. Date: Dec. 4, 2003

(54) Combination immediate release sustained release levodopa/carbidopa dosage forms

(76) Inventors: Chien-Hsuan Han, Sunnyvale, CA (US); Larry Hsu, Santa Clara, CA (US); Richard Ting, Newark, CA (US); Charles Hsiao, Pleasanton, CA (US)

Correspondence Address: Kendrew Colton, Registered Patent Attorney, Fitch, Even, Tabin and Flannery, 1801 K Street, NW., Suite 401L, Washington, DC, 20006-1201, US

(21) Appl. No.: 10/158,412

(22) Filed: May 29, 2002

(51) Int. Cl. A61K 31/198; A61K 9/22

(52) U.S. Cl. 424/468; 514/567

(57) ABSTRACT

Dosage forms containing both immediate release and sustained release components for the treatment of ailments associated with depleted amounts of dopamine in brain tissue of a patient. The dosage forms contain a combination of carbidopa and levodopa.

### Field of the Invention

The present invention relates to dosage forms comprising both immediate release and sustained release components comprising a combination of carbidopa and levodopa for the treatment of ailments associated with depleted amounts of dopamine in a patients brain tissue.

### BACKGROUND OF THE INVENTION

Combinations of carbidopa and levodopa to treat Parkinsons disease are known in the pharmaceutical arts. Several products currently on the North American market, including SINEMET® and SINEMET® CR contain combinations of carbidopa and levodopa in immediate release and controlled release forms respectively.

The carbidopa and levodopa combination are used to treat the symptoms of Parkisons disease which is characterized by abnormally low levels of dopamine. Dopamine is a neurotransmitter having significant influence over the mobility and control of the skeletal muscular system. Patients suffering from Parkinsons disease frequently have periods in which their mobility becomes difficult often resulting in an inability to move.

Administering dopamine is not effective to treat Parkinsons disease because dopamine does not cross the blood brain barrier. To resolve this failure Parkinsons patients are administered levodopa, the metabolic precursor of dopamine. Levodopa crosses the blood brain barrier and is rapidly converted to dopamine, thereby alleviating the symptoms of Parkinsons disease caused by reduced levels of dopamine. Levodopa is problematic because of it's rapid decarboxylation by tissue other than the brain. Thus large doses of levodopa alone are required because only a small portion is transported to the brain unchanged.

Patients treated with levodopa therapy for Parkinsons disease may frequently develop motor fluctuations characterized by end-of-dose failure, peak dose dyskinesia and akinesia. An advanced form of motor fluctuations is known as the “on-off” phenomenon in which the patient suffers from unpredictable swings from mobility to immobility. It is believed that the on-off effect can be minimized in some patients with a treatment regimen which produces narrow ranges of plasma levels of levodopa.

Carbidopa inhibits the decarboxyaltion of levodopa by a patients body tissues outside of the brain. Small does of carbidopa in conjunction with levodopa allow a larger percentage of levodopa to reach the brain unchanged for later conversion to dopamine. There is at least one report that cabidopa reduces the amount of levadopa required to produce a given response by about 75% and when administered in conjunction with levodopa increase plasma levels and the plasma half life of levodopa. The carbidopa and levodopa combination allows for lower does of levodopa with a concordant reduction of side effects.

The carbidopa and levodopa combination is now available in a sustained release compositions which allow for the continuous release of drug over a prolonged period in an attempt to maintain tight levodopa plasma ranges. However, the use of controlled release dosage forms are problematic in that many Parkinson patients wake up in the morning having little or no mobility due to the wearing off of drug from the day or evening before. Such patients usually are unwilling or unable to wait for a controlled release dosage form to deliver the appropriate plasma levels of levodopa.

Combination immediate release and sustained release carbidopa and levodopa dosage forms are described in U.S. Pat. No. 6,238,699 issued May 29, 2001 to Rubin which discloses an immediate release and sustained release carbidopa and levodopa combination product.

There remains a continuing need in the art for immediate release and sustained release carbidopa and levodopa products which will improve the administration of levodopa to Parkisons patients to improve and narrow blood plasma ranges of levodopa and reduce side effects.

### SUMMARY OF THE INVENTION

The present invention is directed to A pharmaceutical dosage form having an immediate release component and a controlled release component comprising: a) an immediate release component comprising a ratio of carbidopa to levodopa of from about 1:2 to about 1:50 such that the in vitro dissolution rate of the immediate release component according to measurements under the USP paddle method of 50 rpm in 900 ml aqueous buffer at pH 4 at 37° C. will be from about 10% to about 90% levodopa released after 30 minutes and from about 50% to about 99% after 1 hour; b) a controlled release component comprising a ratio of cabidopa to levodopa of from about 1:2 to about 1:50 such that the in vitro dissolution rate of the controlled release component according to measurements under the USP paddle method of 50 rpm in 900 ml aqueous buffer at pH 4 at 37° C. will be from about 10% to about 40% levodopa released after 1 hour; from about 25% to about 60% released after 2 hours; from about 40% to about 75% after 4 hours and from about 55% to about 90% after about 6 hours, the in vitro release rate being independent of pH between pH 1.6 and 7.2 and chosen such that the peak plasma level of levodopa obtained in vivo occurs between 1 and 6 hours after administration of the dosage form.

### DESCRIPTION OF THE DRAWINGS

FIGS. 1-8 provide the ingredients and their weights as well as dissolution characteristics of several carbidopa and levodopa combination dosage forms produced and tested according to the present invention.

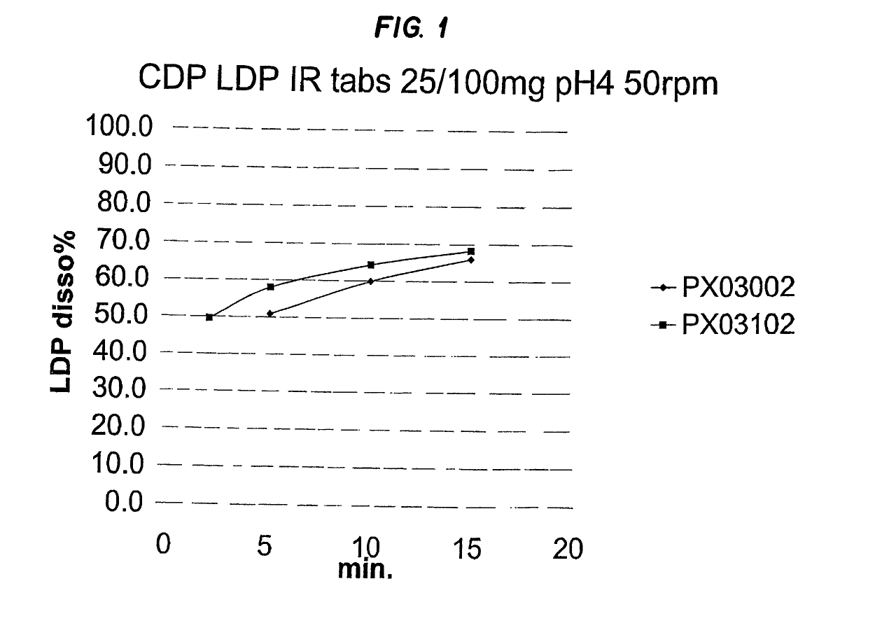

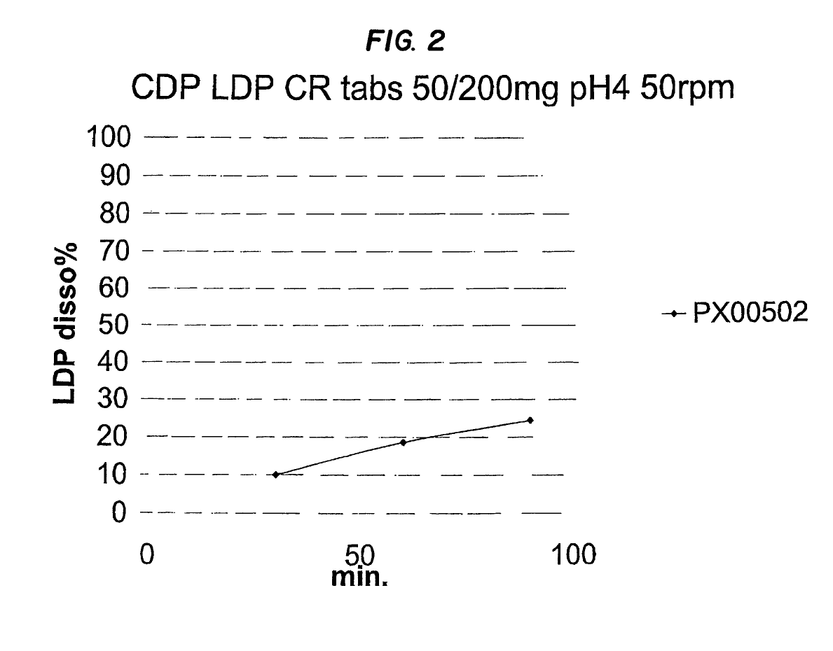

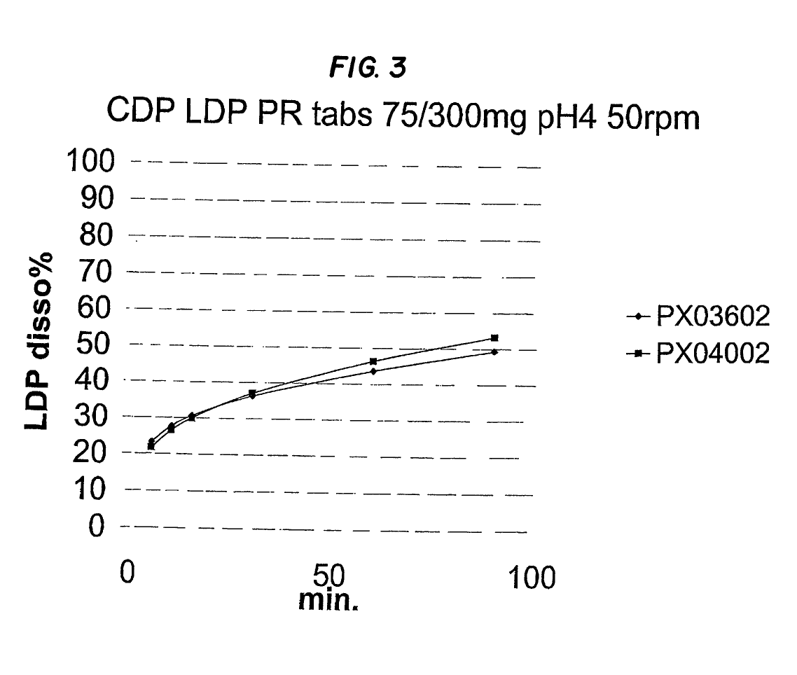

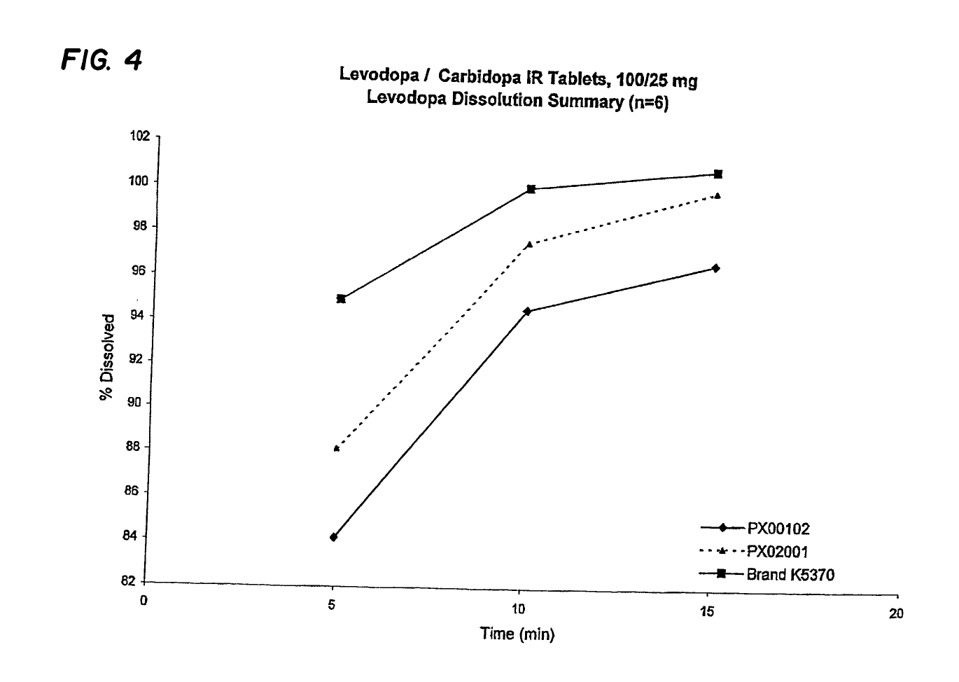

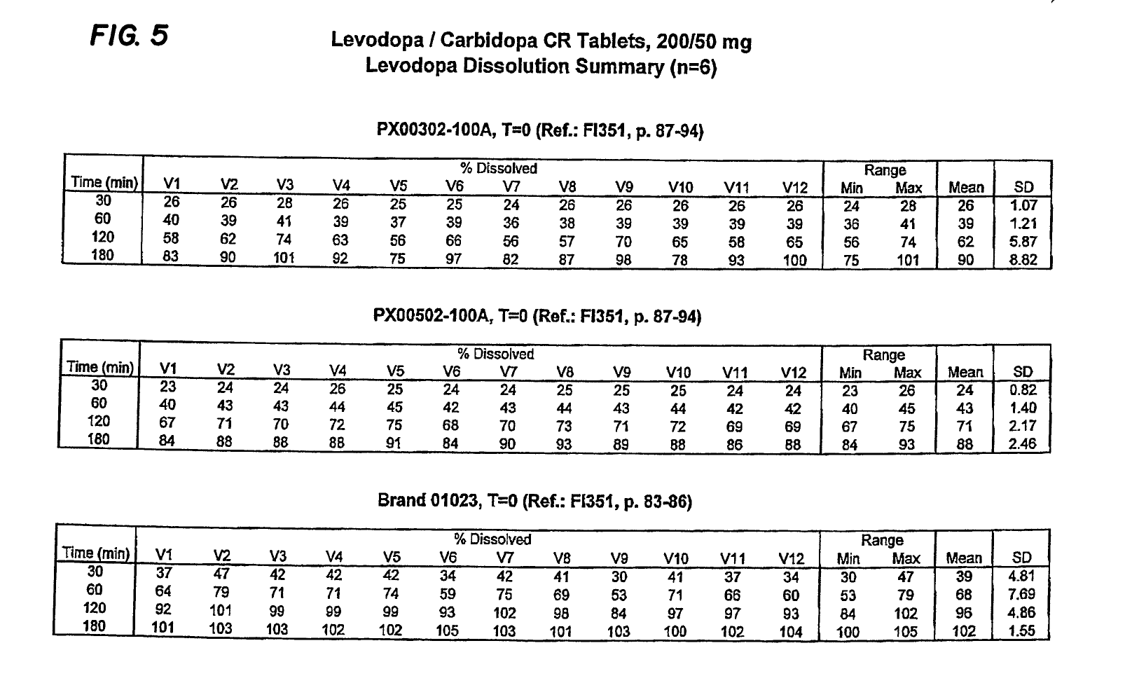

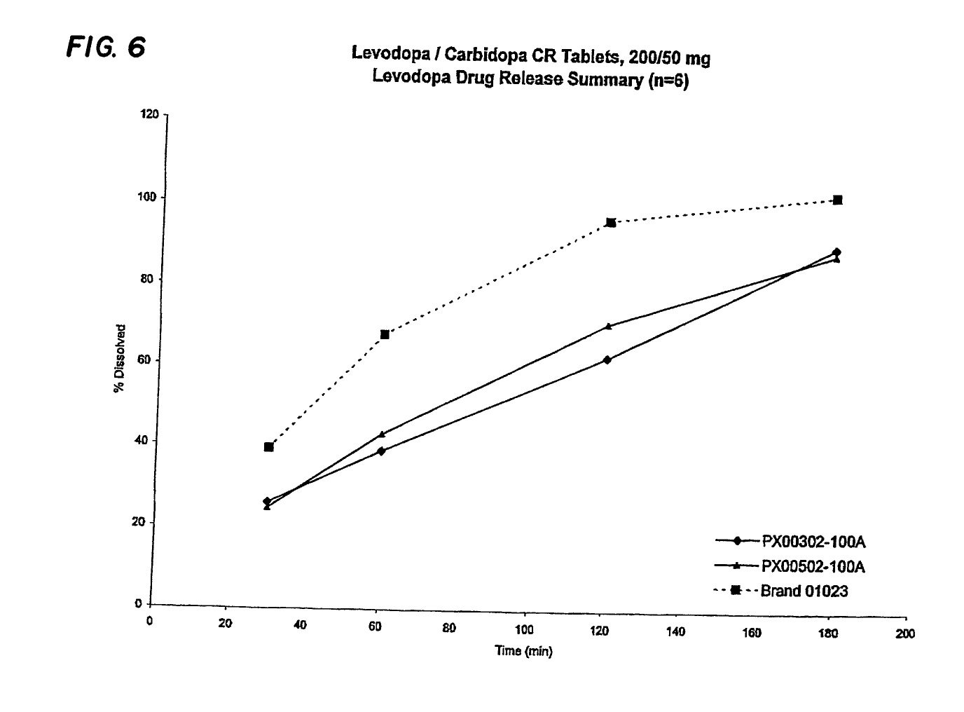

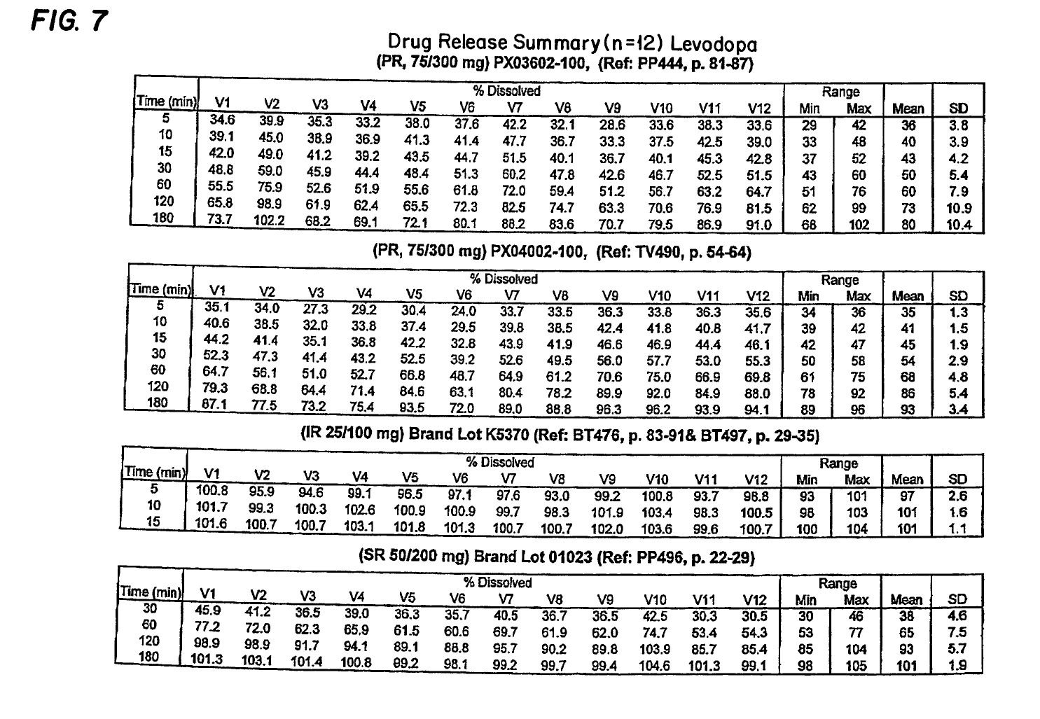

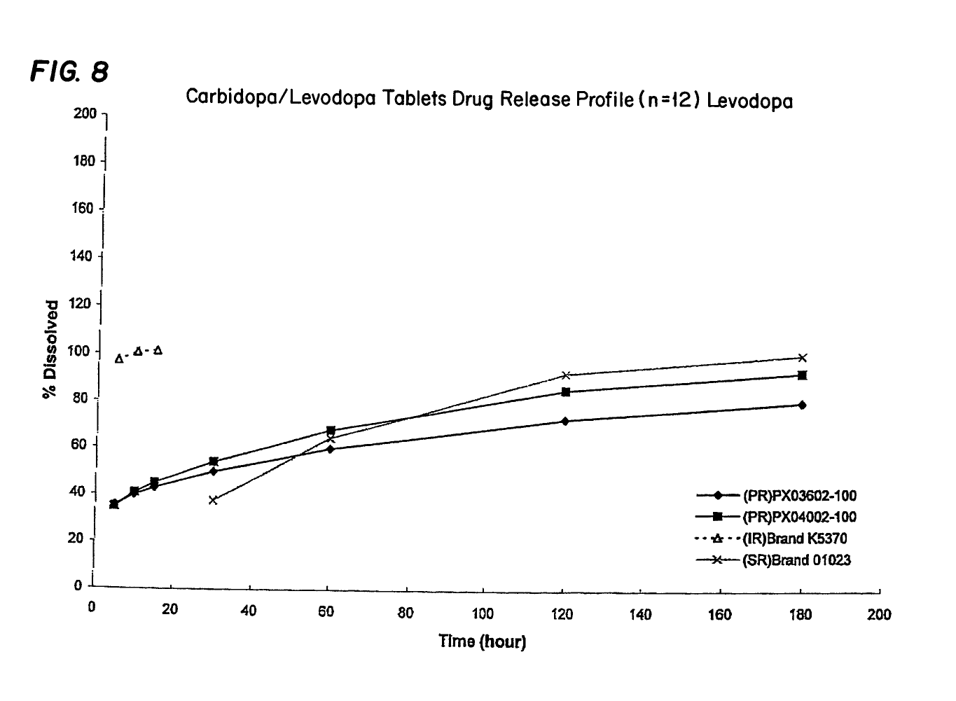

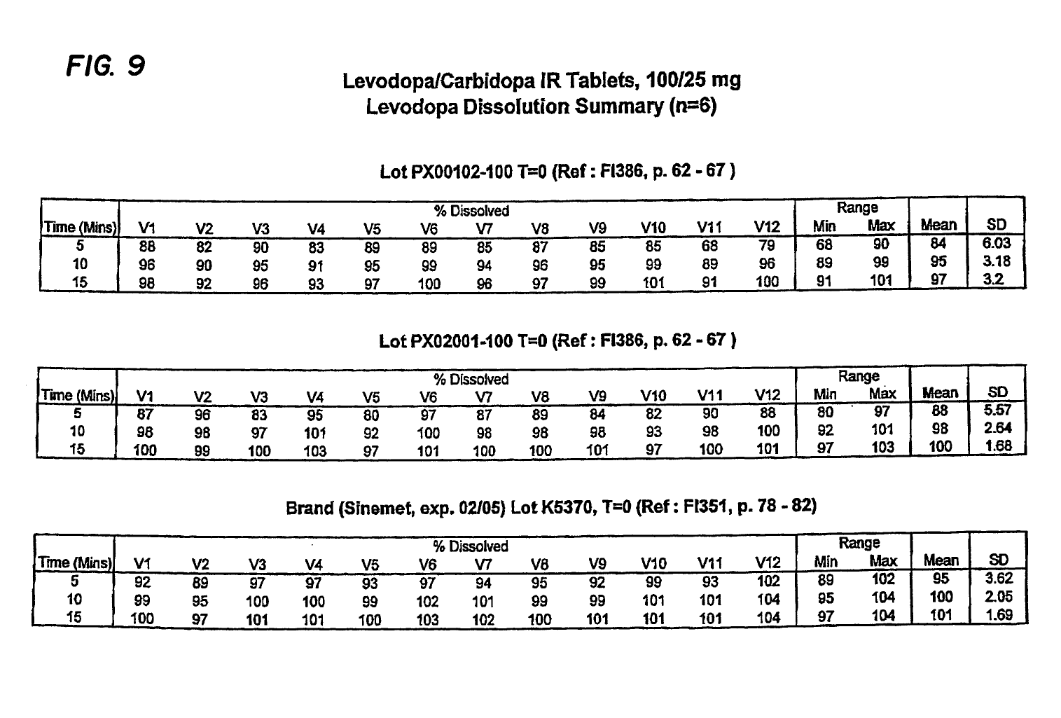

### DETAILED DESCRIPTION OF THE INVENTION

The present invention is directed to a pharmaceutical dosage form having an immediate release component and a controlled release component comprising: a) an immediate release component comprising a ratio of levodopa to carbidopa of from about 1:1 to about 50:1 such that the in vitro dissolution rate of the immediate release component according to measurements under the USP paddle method of 50 rpm in 900 ml aqueous buffer at pH 4 at 37° C. will be from about 10% to about 90% Levodopa released after 30 minutes and from about 50% to about 99% after 1 hour; b) a controlled release component comprising a ratio of levodopa to carbidopa of from about 1:2 to about 1:50 such that the in vitro dissolution rate of the controlled release component according to measurements under the USP paddle method of 50 rpm in 900 ml aqueous buffer at pH 4 at 37° C. will be from about 10% to about 40% levodopa released after 1 hour; from about 25% to about 60% released after 2 hours; from about 40% to about 75% after 4 hours and from about 55% to about 90% after about 6 hours and chosen such that the peak plasma level of levodopa obtained in vivo occurs between 1 and 6 hours after administration of the dosage form.

Total daily dose of the compounds useful according to this invention administered to a host in single or divided doses may be in amounts, for example, of from abut 0.001 to about 100 mg/kg body weight daily and preferably 0.01 to 10 mg/kg/day. Both the levodopa and carbidopa doses will fall within this mg/kg/day dosage range. The relative amounts of levodopa and carbidopa can vary from about 1:1 to about 50:1 in dosage forms according to the present invention. The skilled artisan will appreciate that dosages having an amount of active agent sufficient to treat Parkinsons disease will contain from about 50 mg to about 400 mg of levodopa in combination with from about 10 to about 100 mg of carbidopa. Dosages according to the present invention may also contain from about 100 mg to about 200 mg of levodopa in combination with from about 25 mg to about 50 mg of carbidopa. A preferred dosage form contains 100 mg of levodopa and 25 mg of carbidopa.

Dosage unit compositions may contain such amounts of such submultiples thereof as may be used to make up the daily dose. It will be understood, however, that the specific dose level for any particular patient will depend upon a variety of factors including the body weight, general health, sex, diet, time and route of administration, rates of absorption and excretion, combination with other drugs and the severity of the particular disease being treated.

For purposes of the present invention, the term patient means any mammal including humans.

Actual dosage levels of active ingredient in the compositions of the invention may be varied so as to obtain an amount of active ingredient that is effective to obtain a desired therapeutic response for a particular composition and method of administration. The selected dosage level therefore depends upon the desired therapeutic effect, on the route of administration, on the desired duration of treatment and other factors.

The dosage forms of the present invention are designed to administer active agent according to two separate profiles. The first profile is an immediate release burst of the combination of active ingredients to provide short term relief from symptoms via quick onset of effective blood plasma levels of active agent. Such short term but quick term release is such that the in vitro dissolution rate of the immediate release component according to measurements under the USP paddle method of 50 rpm in 900 ml aqueous buffer at pH 4 at 37° C. will be from about 10% to about 90% levodopa released after 30 minutes and from about 50% to about 99% after 1 hour. USP refers to the United States Pharmacopeia.

The second profile is a controlled release profile in which the combination of active ingredients is released slowly over time to provide a blood plasma level effective to alleviate the symptoms of Parkinsons disease over a prolonged period. The controlled release profile may be over a period of 6, 12, or 24 hours. The controlled release profile of the present invention is such that the in vitro dissolution rate of the controlled release component according to measurements under the USP paddle method of 50 rpm in 900 ml aqueous buffer at pH 4 at 37° C. will be from about 10% to about 40% levodopa released after 1 hour; from about 25% to about 60% released after 2 hours; from about 40% to about 75% after 4 hours and from about 55% to about 90% after about 6 hours and chosen such that the peak plasma level of levodopa obtained in vivo occurs between 1 and 6 hours after administration of the dosage form.

The active ingredients of the present invention may be mixed with pharmaceutically acceptable carriers, diluents, adjuvants, excipients, or vehicles, such as preserving agents, fillers, polymers, disintegrating agents, wetting agents, emulsifying agents, suspending agents, sweetening agents, flavoring agents, perfuming agents, lubricating agents and dispensing agents, depending on the nature of the mode of administration and dosage forms. Such ingredients including pharmaceutically acceptable carriers and excipients that may be used to formulate oral dosage forms are described in the Handbook of Pharmaceutical Excipients, American Pharmaceutical Association (1986), incorporated by reference herein and include water, ethanol, polyols, suitable mixtures thereof, vegetable oils. Examples of excipients include starch, pregelatinized starch, Avicel, lactose, milk sugar, sodium citrate, calcium carbonate, dicalcium phosphate and lake blend. Examples of disintegrating agents include starch, alginic acids and certain complex silicates. Examples of lubricants include magnesium stearate, sodium lauryl sulphate, talc, as well as high molecular weight polyethylene glycols.

Dosage forms according to the invention can be made according to well known methods in the art.

Matrix Dosage Forms

Dosage forms according to the present invention may be in the form of coated matrices. The coating will contain the immediate release combination of levodopa and carbidopa and the matrix itself can contain the controlled release combination of levodopa and carbidopa.

The term matrix is given it well known meaning in the pharmaceutical arts, that is a solid material having active agent incorporated therein. Upon exposure to a dissolution media, channels are formed in the solid material so that active agent can escape.

The skilled artisan will appreciate that the matrix material can be chosen from a wide variety of materials which can provide the desirable dissolution profiles. Materials include one or more cellulose ethers such as hydroxypropyl methyl cellulose, hydroxy propyl cellulose, ethyl cellulose, polyvinyl pyrrolidone, fats, methacrylic acid copolymers and methacrylate polymers to name a few.

Methods of making matrix dosages are well known in the art and any known method of making such dosages which will yield the desired immediate release and controlled release dissolution profiles can be used. One method involves the mixture of the levodopa and carbidopa combination with a solid polymeric material and one or more pharmaceutically acceptable excipients which are then blended and compressed in controlled release tablet cores. Such tablet cores can be used for further processing as bi-layer tablets, press coated tablets or film coated tablets.

A coating containing the immediate release levodopa carbidopa combination can be added to the outside of the controlled release tablet cores to produce a final dosage form according to the present invention. Such coating can be prepared by mixing a combination of levodopa and carbidopa with PVP 29/32 or HPMC and water/isopropyl alcohol and triethyl acetate. Such an immediate release coating can be spray coated onto the tablet cores.

The instant release coating may also be applied using the press-coating process with a blend consisting of levodopa and carbidopa 80% w/lactose and hydroxypropyl methylcellulose type 2910.

In addition, the formulation of respective release compartments can occur by appropriate granulation methods. In wet granulation, solutions of the binding agent (polymer) are added with stirring to the mixed powders. The powder mass is wetted with the binding solution until the mass has the consistency of damp snow or brown sugar. The wet granulated material is forced through a sieving device. Moist material from the milling step is dried by placing it in a temperature controlled container. After drying, the granulated material is reduced in particle size by passing through a sieving device. Lubricant is added, and the final blend is then compressed.

In fluid-bed granulation, particles of inert material and/or active agent are suspended in a vertical column with a rising air stream. While the particles are suspended, the common granulating materials in solution are sprayed into the column. There is a gradual particle buildup under a controlled set of conditions resulting in tablet granulation. Following drying and the addition of lubricant, the granulated material is ready for compression.

In dry-granulation, the active agent, diluent, and lubricant are blended and compressed into large tablets. The compressed large tablets are comminuted through the desirable mesh screen by sieving equipment. Some more lubricant is added to the granulated material and blended gently. The material is then compressed into tablets.

Immediate Release Particles

The immediate release/controlled release dosage forms of the present invention can also take the form of pharmaceutical particles. The dosage forms would include immediate release particles in combination with controlled release particles in a ratio sufficient to deliver the desired dosages of active agents. The controlled release particles can be produced by coating the immediate release particles.

The particles according to the present invention can be produced according to any of a number of well known methods for making particles. The immediate release particles would comprise the active agent combination and a disintegrant. Suitable disintegrents include starch, low-substitution hydroxypropyl cellulose, croscarmellose sodium, calcium carboxymethyl cellulose, hydroxypropyl starch and microcrystalline cellulose for example.

In addition to the above ingredients, a sustained release matrix may also contain suitable quantities of other materials, e.g., diluents, lubricants, binders, granulating aids, colorants, flavorants and glidants that are conventional in the pharmaceutical art. The quantities of these additional materials will be sufficient to provide the desires effect to the desired formulation. In addition to the above ingredients, a sustained release matrix incorporating particulates may also contain suitable quantities of other materials, e.g. diluents, lubricants, binders, granulating aids, colorants, flavorants and glidants that are conventional in the pharmaceutical art in amounts up to about 75% by weight of the particulate if desired.

Particles according to the present invention can assume any standard structure known in the pharmaceutical arts. Such structures include, for example, matrix particles, nonpareil cores having a drug layer and active or inactive cores having multiple layers thereon. A controlled release coating can be added to any of these structures to create a controlled release particle.

The term particle as used in the present invention and means a granule having a diameter of between 0.01 mm and 5.0, more preferably between 0.1 mm and 2.5 mm or even more preferably between 0.5 mm and 2 mm. The skilled artisan will appreciate that particles according to the present invention can be any geometrical shape within this size range.

The release of the therapeutically active agent from the controlled release formulation of the present invention can be further influenced, i.e., adjusted to a desired rate, by the addition of one or more release-modifying agents.

The release-modifying agents may be organic or inorganic, and include materials that can be dissolved, extracted or leached from the coating in the environment of use. The pore-formers may comprise one or more hydrophilic materials such as hydroxypropylmethylcellulose.

The release-modifying agent may also comprise a semi-permeable polymer.

In certain preferred embodiments, the release-modifying agent is selected from hydroxypropylmethyclcellulose, lactose, metal stearates, and mixtures of any of the foregoing.

In one preferred embodiment, oral dosage forms are prepared to include an effective amount of particulates described above within a capsule. For example, a plurality of the melt-extruded multiparticulates may be placed in a gelatin capsule in an amount sufficient to provide an effective sustained release dose when ingested and contacted by gastric fluid.

In another preferred embodiment, a suitable amount of the particles are compressed into an oral tablet using conventional tableting equipment using standard techniques. Techniques and compositions for making tablets (compressed and molded), capsules (hard and soft gelatin) and pills are also described in Remington's Pharmaceutical Sciences, (Arthur Osol, editor), 1553-1593 (1980), incorporated by reference herein.

The particles of the present invention can be made by mixing the relevant ingredients and granulating the mixture. The resulting particles are dried and screened and all particles having the desirable size are employed for purposes of the present invention.

Controlled Release Particles

When controlled release particles according to the present invention, the immediate release particles can be coated with a controlled release coating in order to change the release rate to obtain the dissolution rates of the present invention.

The controlled release particles of the present invention slowly release the combination of levodopa and carbidopa when ingested and exposed to gastric fluids, and then to intestinal fluids. The controlled release profile of the formulations of the invention can be altered, for example, by increasing or decreasing the thickness of the retardant coating, i.e., by varying the amount of overcoating.

A plurality of the resultant solid controlled release particles may thereafter be placed in a gelatin capsule in an amount sufficient to provide an effective controlled release dose when ingested and contacted by an environmental fluid, e.g., gastric fluid or dissolution media.

The particles may be overcoated with an aqueous dispersion of a hydrophobic or hydrophilic material to modify the release profile. The aqueous dispersion of hydrophobic material preferably further includes an effective amount of plasticizer, e.g. triethyl citrate. Preformulated aqueous dispersions of ethylcellulose, such as Aquacoat® or Surelease®, may be used. If Surelease® is used, it is not necessary to separately add a plasticizer.

The hydrophobic material may be selected from the group consisting of alkylcellulose, acrylic and methacrylic acid polymers and copolymers, shellac, zein, hydrogenated castor oil, hydrogenated vegetable oil, or mixtures thereof. In certain preferred embodiments of the present invention, the hydrophobic material is a pharmaceutically acceptable acrylic polymer, including but not limited to acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylicacid alkylamine copolymer, poly(methyl methacrylate), poly(methacrylic acid)(anhydride), polymethacrylate, polyacrylamide, poly(methacrylic acid anhydride), and glycidyl methacrylate copolymers. In alternate embodiments, the hydrophobic material is selected from materials such as one or more hydroxyalkycelluloses such as hydroxypropylmethycellulose and mixtures of the foregoing. The at least one hydroxyalkyl cellulose is preferably a hydroxy (C₁ to C₆) alkyl cellulose, such as hydroxypropylcellulose, hydroxypropylmethylcellulose, especially, hydroxyethylcellulose. The amount of the at least one hydroxyalkyl cellulose in the present oral dosage form will be determined, inter alia, by the precise rate of active agents desired and may vary between 1% and about 80%.

In embodiments of the present invention where the coating comprises an aqueous dispersion of a hydrophobic polymer, the inclusion of an effective amount of a plasticizer in the aqueous dispersion of hydrophobic polymer will further improve the physical properties of the film. For example, because ethylcellulose has a relatively high glass transition temperature and does not form flexible films under normal coating conditions, it is necessary to plasticize the ethylcellulose before using the same as a coating material. Generally, the amount of plasticizer included in a coating solution is based on the concentration of the film-former, e.g., most often form about 1 to about 50 percent by weight of the film-former. Concentration of the plasticizer, however, can only be properly determined after careful experimentation with the particular coating solution and method of application.

Examples of suitable plasticizers for ethylcellulose include water insoluble plasticizers such as dibutyl sebacate, diethyl phthalate, triethyl citrate, tributyl citrate, and triacetin, although it is possible that other water-insoluble plasticiers (such as acetylated monoglycerides, phthalate esters, castor oil, etc.) may be used. Triethyl citrate is an especially preferred plasticizer for the aqueous dispersions of ethyl cellulose of the present invention.

Examples of suitable plasticizer for the acrylic polymers of the present invention include, but are not limited to citric acid esters such as triethyl citrate NF XVI, tributyl citrate, dibutyl phthalate, and possibly 1,2-propylene glycol. Other plasticizers which have proved to be suitable for enhancing the elasticity of the films formed from acrylic films such as Eudragit® RL/RS lacquer solutions include polyethylene glycols, propylene glycol, diethyl phthalate, castor oil, and triacetin. Triethyl citrate is an especially preferred plasticizer for the aqueous dispersions of ethyl cellulose of the present invention.

It has further been found that addition of a small amount of talc reduces the tendency of the aqueous dispersion to stick during processing, and acts a polishing agent.

One commercially-available aqueous dispersion of ethylcellulose is Aquacoat® is prepared by dissolving the ethylcellulose in a water-immiscible organic solvent and then emulsifying the same in water in the presence of a surfactant and a stabilizer. After homogenization to generate submicron droplets, the organic solvent is evaporated under vacuum to form a pseudolatex. The plasticizer is not incorporated in the pseudolatex during the manufacturing phase. Thus, prior to using the same as a coating, it is necessary to intimately mix the Aquacoat® with a suitable plasticizer prior to use.

Another aqueous dispersion of ethylcellulose is commercially available as Surelease® (Colorcon, Inc., West Point, Pa., U.S.A.). This product is prepared by incorporating plasticizer into the dispersion during the manufacturing process. A hot melt of a poymer, plasticizer (dibutyl sebacate), and stabilizer (oleic acid) is prepared as a homogeneous mixture, which is then diluted with an alkaline solution to obtain an aqueous dispersion which can be applied directly onto substrates.

In one preferred embodiment, the acrylic coating is an acrylic resin lacquer used in the form of an aqueousdispersion, such as that which is commercially available from Rohm Pharma under the Tradename Eudragit®. In further preferred embodiments, the acrylic coating comprises a mixture of two acrylic resin lacquers commercially available from Rohm Pharma under the Tradenames Eudragit® RL 30 D and Eudragit® RS 30 D, respectively. Eudragit® RL 30 D and Eudragit® RS 30 are copolymers of arcrylic and methacrylic esters with a low content of quaternary ammonium groups, the molar ratio of ammonium groups to the remaining neutral (meth)acrylic esters being 1:20 in Eudragit® RL 30 and 1:40 in Eudragit® RS 30 D. The mean molecular weight is about 150,000. The code designations RL (high permeability) and RS (low permeability) refer to the permeability properties of these agents. Eudragit® RL/RS mixtures are insoluble in water and in digestive fluids. However, coating formed from the same are swellable and permeable in aqueous solutions and digestive fluids.

The Eudragit® RL/RS dispersions of the present invention may be mixed together in any desired ratio in order to ultimately obtain a controlled-release formulation having a desirable dissolution profile. Desirable controlled-release formulations may be obtained, for instance, from a retardant coating derived from 100% Eudragit® RL, 50% Eudragit® RL and 50% Eudragit® RS, and 10% Eudragit® RL: Eudragit® 90% RS. Of course, one skilled in the art will recognize that other acrylic polymers may also be used, such as, fro example, Eudragit® L.

In addition to modifying the dissolution profile by altering the relative amounts of different acrylic resin lacquers, the dissolution profile of the ultimate product may also be modified, for example, by increasing or decreasing the thickness of the retardant coating.

In preferred embodiments of the present invention directed to the acrylic coating, the stabilized product is obtained by subjecting the coated substrate to oven curing at a temperature above the Tg of the plasticized acrylic polymer for the required time period, the optimum values for temperature and time for the particular formulation being determined experimentally. In certain embodiments of the present invention, the stabilized product is obtained via an oven curing conducted at a temperature of about 45° C. for a time period from about 24 to about 48 hours. It is also comtemplated that certain products coated with the controlled-release coating of the present invention may require a curing time longer than 24 hours, e.g., from about 24 to about 48 hours, or even 60 hours or more.

The coating solutions of the present invention preferably contain, in addition to the film-former, plasticizer, and solvent system (i.e., water), a colorant to provide elegance and product distinction. Color may be added to the solution of the therapeutically active agent instead, or in addition to the aqueous dispersion of hydrophobic material. For example, color may be added to Aquacoat® via the use of alcohol or propylene glycol based color dispersions, milled aluminum lakes and opacifiers such as titanium dioxide by adding color with shear to water soluble polymer solution and then using low shear to the plasticized Aquacoat®. Alternatively, any suitable method of providing color to the formulations of the present invention may be used. Suitable ingredients for providing color to the formulation when an aqueous dispersion of an acrylic polymer is used include titanium dioxide and color pigments, such as iron oxide pigments. The incorporation of pigments, may, however, increase the retard effect of the coating.

Spheroids or beads coated with the therapeutically active agents are prepared, e.g., by dissolving the therapauetically active agents in water and then spraying the solution onto a substrate, for example, nu pariel 18/20 beads, using a Wuster insert. Optionally, additional ingredients are also added prior to coating the beads in order to assist the binding of the active agents to the beads, and/or to color the solution, etc. For example, a product which includes hydroxypropylmethycellulose, etc. with or without colorant (e.g., Opadry®, commercially available from Coloron, Inc.) may be added to the solution and the solution mixed (e.g., for about 1 hour) prior to application of the same onto the beads. The resultant coated substrate, in this example beads, may then be optionally overcoated with a barrier agent, to separate the therapeutically active agent from the hydrophobic controlled release coating. An example of a suitable barrier agent is one which comprises hydroxypropyhnethylcellulose. However, any film-former known in the art may be used. It is preferred that the barrier agent does not affect the dissolution rate if the final product.

Press Coated, Pulsatile Dosage Form

In another embodiment of the present invention the carbidopa/levodopa combination is administered via a press coated pulsatile drug delivery system suitable for oral administration with an sustained release compartment, which contains a compressed blend of an active agent and one or more polymers, substantially enveloped by an immediate release compartment, which contains a compressed blend of the active agent and hydrophilic and hydrophobic polymers.

The immediate-release compartment preferably comprises a compressed blend of active agent and one or more polymers with disintegration characteristics, which upon exposure to the aqueous medium, would break apart quickly.

The extended-release compartment preferably comprises a combination of hydrophilic and hydrophobic polymers. In this embodiment, once administered, the hydrophilic polymer dissolves away to weaken the structure of the extended-release compartment, and the hydrophobic polymer retards the water penetration and helps to maintain the shape of the drug delivery system.

In accordance with the present invention, the term “polymer” includes single or multiple polymeric substances, which can swell, gel, degrade or erode on contact with an aqueous environment (e.g., water), such as one or more of alginic acid, carboxymethylcellulose calcium, carboxymethylcellulose sodium, colloidal silicon dioxide, croscarmellose sodium, crospovidone, guar gum, magnesium aluminum silicate, methylcellulose, microcrystalline cellulose, polacrilin potassium, powdered cellulose, pregelatinized starch, sodium alginate, sodium starch glycolate, starch, ethylcellulose, gelatin, hydroxyethyl cellulose, hydroxypropyl cellulose, hydroxypropyl methylcellulose, polymethacrylates, povidone, pregelatinized starch, shellac, and zein, and combinations thereof.

The “hydrophilic polymers” of the present invention include one or more of carboxymethylcellulose, natural gums such as guar gum or gum acacia, gum tragacanth, or gum xanthan, hydroxyethyl cellulose, hydroxypropyl cellulose, hydroxypropyl methylcellulose, methylcellulose, and povidone of which hydroxypropyl methylcellulose is further preferred. “Hydrophilic polymers” also include sodium carboxymethycellulose, hydroxymethl cellulose, polyethelene oxide, hydroxyethyl methyl cellulose, carboxpoylmethylene, polyethelene glycol, alginic acid, gelatin, polyvinyl alcohol, polyvinylpyrrolidones, polyacrylamides, polymethacrylamides, polyphosphazines, polyoxazolidines, poly(hroxyalky)carboxylic acids), an alkali metal or alkaline earth metal, carageenate alginates, ammonium alginate, sodium alganate, or mixtures thereof. The hydrophilic polymer can be any hydrophilic polymer which will achieve the goals of the present invention including, but not limited to, one or more polymers selected from.

The hydrophobic polymer of the drug delivery system can be any hydrophobic polymer which will achieve the goals of the present invention including, but not limited to, one or more polymers selected from carbomer, carnauba wax, ethylcellulose, glyceryl palmitostearate, hydrogenated castor oil, hydrogenated vegetable oil type 1, microcrystalline wax, polacrilin potassium, polymethacrylates, or stearic acid, of which hydrogenated vegetable oil type 1 is further preferred. The “hydrophobic polymers” of the present invention may also be selected from the group consisting of a pharmaceutically acceptable acrylic polymer, including, but not limited to, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methyacrylate), poly(methyl methacrylate)copolymer, polyacrylamide, aminoalkyl methacrylate copolymer, poly(methacrylic acid anhydride), and glycidyl methacrylate copolymers. Additionally the acrylic polymers may be cationic, anionic, or non-ionic polymers and may be acrylates, methacrylates, formed of methacrylic and or methacrylic acid esters. The polymers may also be dependent.

The present invention also encompasses a method for preparing a press coated, pulsatile drug delivery system suitable for oral administration including the steps of combining an effective amount of an active agent, or a pharmaceutically acceptable salt thereof, and a polymer to form an immediate-release compartment; combining an effective amount of an active agent, or a pharmaceutically acceptable salt thereof, and a combination of hydrophilic and hydrophobic polymers to form an extended-release compartment; and press coating the extended-release compartment to substantially envelop the immediate-release compartment.

A preferred embodiment further includes the steps of combining an effective amount of an active agent, or a pharmaceutically acceptable salt thereof, and a polymer to form an instant-release compartment, and press coating the instant-release compartment to substantially envelop the extended-release compartment.

In another preferred embodiment, the combining steps can be done by blending, wet granulation, fluid-bed granulation, or dry granulation according to methods recognized in the art.

The term “substantially envelop is intended to define the total or near-total enclosure of a compartment. Such an enclosure includes, preferably at least 80% enclosure, more preferably at least 90% enclosure, and preferably at least 99% enclosure.

### EXAMPLES

Without further elaboration, it is believed that one skilled in the art, using the preceding description, can utilize the present invention to the fullest extent. The following examples are illustrative only, and not limiting of the remainder of the disclosure in any way whatsoever.

### Example 1

The method below was employed to obtain a press coated, pulsatile drug delivery system, the composition of which is set forth in the following tables.

TABLE 1

| | Quantity/Tablet | | | |
|---|---|---|---|---|
| | Example #1 | | Example #2 | |
| | RT-010 (press- | | RT-01 1 (press | |
| | coated w/o instant- | | coated w/ instant- | |
| | release coating) | | release coating) | |
| Immediate-Release (IR) Compartment | | | | |
| Levodopa/carbidopa 4:1 ratio 80% w/ | 50.0 | mg | 50.0 | mg |
| lactose | | | | |
| Croscarmellose sodium | 1.6 | mg | 1.6 | mg |
| Microcrystalline cellulose | 26.8 | mg | 26.8 | mg |
| Colloidal silicon dioxide | 0.8 | mg | 0.8 | mg |
| Magnesium stearate | 0.8 | mg | 0.8 | mg |
| Total: | 80.0 | mg | 80.0 | mg |
| IR Compartment Plus Extended- | | | | |
| Release (ER) Compartment | | | | |
| IR Compartment | 80.0 | mg | 80.0 | mg |
| Levodopa/carbidopa 4:1 ratio 80% w/ | 37.5 | mg | 18.8 | mg |
| lactose | | | | |
| Hydroxypropyl methylcellulose type 2208 | 61.6 | mg | 61.6 | mg |
| Microcrystalline cellulose | 70.3 | mg | 89.0 | mg |
| Hydrogenated vegetable oil type 1 | 46.2 | mg | 46.2 | mg |
| Colloidal silicon dioxide | 2.2 | mg | 2.2 | mg |
| Magnesium stearate | 2,.2 | mg | 2.2 | mg |
| Total: | 300.0 | mg | 300.00 | mg |
| IR Compartment Plus ER | | | | |
| Compartment Plus | | | | |
| Instant-Release Compartment | | | | |
| ER Compartment Plus ER Compartment | | | 300.0 | mg |
| Levodopa/carbidopa 4:1 ratio 80% w/ | | | 18.7 | mg |
| lactose | | | | |
| Hydroxypropyl methylcellulose type 2910 | | | 1.9 | mg |
| Total: | | | 320.6 | mg |

TABLE 2
EXCIPIENT RANGE

| | Quantity/tablet | | | |
|---|---|---|---|---|
| | Example #1 | | | |
| | RT-010 (press | | | |
| | coated w/o | | | |
| | instant-release | | | |
| | coating) | | Percent | Range |
| Immediate-Release Compartment | | | | |
| Levodopa/carbidopa 4:1 ratio 80% w/ | 50.0 | mg | 62.5% | |
| lactose | | | | |
| Croscarmellose sodium | 1.6 | mg | 2.0% | 0.5-10.0% |
| Microcrystalline cellulose | 26.8 | mg | 33.5% | 18.0-36.0% |
| Colloidal silicon dioxide | 0.8 | mg | 1.0% | 0.5-2.0% |
| Magnesium stearate | 0.8 | mg | 1.0% | 0.5-2.0% |
| Total: | 80.0 | mg | | |
| Extended-Release Compartment | | | | |
| Levodopa/carbidopa 4:1 ratio 80% w/ | 37.5 | mg | 17.0% | |
| lactose | | | | |
| Hydroxypropyl methylcellulose type 2208 | 61.6 | mg | 28.0% | 15.0-40.0% |
| Microcrystalline cellulose | 70.3 | mg | 32.0% | 8.0-57.0% |
| Hydrogenated vegetable oil type 1 | 46.2 | mg | 21.0% | 10.0-30.0% |
| Colloidal silicon dioxide | 2.2 | mg | 1.0% | 0.5-2.0% |
| Magnesium stearate | 2.2 | mg | 1.0% | 0.5-2.0% |
| Total: | 220.0 | mg | | |

Appropriate weights of levodopa and carbidopa are intimately mixed for use in preparing immediate release and controlled release components of the present invention.

Immediate-Release Compartment. The active agents are first mixed with silicon dioxide in a Patterson-Kelley V-blender for 10 minutes, then microcrystalline cellulose and crosscarmellulose sodium are added and blended for 10 more minutes. Finally, magnesium stearate is added to the blender and mixed for another 10 minutes. The powder blend is then compressed using a Manesty Dry-cota with 0.2031″ in diameter, round, flat-face punch and die set. The hardness of the tablets are maintained at 4±2 kp.

Immediate-Release Compartment Plus Controlled-Release Compartment

The active agents are first mixed with silicon dioxide in a Patterson-Kelley V-blender for 10 minutes, then hydroxypropyl methylcellulose 2208 and microcrystalline cellulose are added and blended for 10 more minutes. Finally, hydrogenated vegetable oil and magnesium stearate are added to the blender and mixed for another 10 minutes. The core tablets are press-coated using the Manesty Dry-cota with 0.3600″ in diameter, round, shallow concave punch and die set. The hardness of the tablets are maintained at 12±4 kp.

### Example 2

Immediate-Release Compartment Plus Extended-Release Compartment Plus Instant-Release Compartment

The method of manufacture for the controlled-release tablets is the same as described in Example 1. The application of the instant-release compartment was done by charging the extended-release tablets into a perforated pan coater or a fluidized particle coater and coating the tablet cores with a solution consisting of levodopa and carbidopa 80% w/lactose and hydroxypropyl methylcellulose type 2910.

### Example 3

Example 3 employs the ingredients and amounts of ingredients listed in Table 2 below.

TABLE 2
Summary of CDP/LDP PR tabs 75/300

| PR tablets PX03602 is the combination of PX0502(CR) and PX03102 | | | |
|---|---|---|---|
| PR tablets PX04002 is the combination of PX0502(CR) and PX03002 | | | |
| | | per talbet | |
| | (w/w) % | (w/w) % | amount |
| CR | | | |
| PX00502 | | | |
| Carbidopa | 14 | 18 | 53.8 |
| Levodopa | 51.9 | 67 | 200.1 |
| Klucel | 10 | 12.9 | 38.5 |
| Lake blend | 0.2 | 0.3 | 0.9 |
| Mg stearate | 1.4 | 1.8 | 5.4 |
| Total | 77.5 | 100 | 298.7 |
| IR | | | |
| PX03002 | | | |
| Carbidopa | 10.21 | 11.3 | 27 |
| Levodopa | 37.84 | 41.9 | 100 |
| Avicel | 29.95 | 33.2 | 79.2 |
| Starch | 10 | 11.1 | 26.5 |
| Acdisol | 0.75 | 0.8 | 1.9 |
| Mg stearate | 1.5 | 1.7 | 3.8 |
| Total | 90.25 | 100 | 238.4 |
| PX03102 | | | |
| Carbidopa | 10.21 | 9.3 | 26.9 |
| Levodopa | 37.84 | 34.6 | 100.1 |
| Avicel | 29.95 | 27.4 | 79.3 |
| Starch | 30 | 27.4 | 79.3 |
| Mg Stearate | 1.5 | 1.3 | 3.8 |
| Total | 109.5 | 100 | 289.4 |

For each batch, whether 502, 3002 or 3102, the same procedure is used as follows: All ingredients, except magnesium stearate are weighed and mixed thoroughly. The mixed ingredients are added to a high shear granulator and mixed for 5 minutes, with an impeller speed of 5 and a chopper speed of 4. Deionized water is employed as the granulating agent. Granules so made are dried in an oven overnight and then screened through a #20 mesh (US standard). Oversize granules are milled, screened with the process repeated until all particles can be screened through a #20 mesh.

The magnesium stearate is added to the screened particles and mixed thoroughly.

The resulting mixture can then be used for different types of dosage forms as set out in examples 4 and 5.

### Example 4

Particles produced in example 4, lot 3102 are segregated into two equal portions of 125 g each. One portion is coated in a fluidized pan with mixture of 24.25 g of PVP 29/32, 1000 g of deionized water and isopropyl alcohol (15%) and 0.75 g of triethyl acetate. The particles are dried and thoroughly mixed with the uncoated particles. The particle mixture is then loaded into immediate release gelatin capsules.

### Example 5

Particles produced according to lots 3002 and 502 are loaded into the two separate hoppers of a dual layer tablet punch. The punch is actuated and two layer tablets are produced.

Other objects, features and advantages of the present invention will become apparent from the following detailed description. The detailed description and the specific examples, however, indicate only preferred embodiments of the invention.

Various changes and modifications within the spirit and scope of the invention will become apparent to those skilled in the art from this detailed description.

I (We) claim:

1. A pharmaceutical dosage form having an immediate release component and a controlled release component comprising:
a) an immediate release component comprising a ratio of carbidopa to levodopa of from about 1:2 to about 1:50 such that the in vitro dissolution rate of the immediate release component according to measurements under the USP paddle method of 50 rpm in 900 ml aqueous buffer at pH 4 at 37° C. is from about 10% to about 90% Levodopa released after 30 minutes and from about 50% to about 99% after 1 hour;
b) a controlled release component comprising a ratio of cabidopa to levodopa of from about 1:2 to about 1:50 such that the in vitro dissolution rate of the controlled release component according to measurements under the USP paddle method of 50 rpm in 900 ml aqueous buffer at pH 4 at 37° C. is from about 10% to about 40% levodopa released after 1 hour; from about 25% to about 60% released after 2 hours; from about 40% to about 75% after 4 hours and from about 55% to about 90% after about 6 hours, the in vitro release rate being independent of pH between pH 1.6 and 7.2 and chosen such that the peak plasma level of levodopa obtained in vivo occurs between 1 and 6 hours after administration of the dosage form.

2. A pharmaceutical dosage form according to claim 1 wherein the immediate release component comprises a ratio of carbidopa to levodopa of from about 1:3 to about 1:20.

3. A pharmaceutical dosage form according to claim 1 wherein the immediate release component comprises a ratio of carbidopa to levodopa of from about 1:4 to about 1:16.

4. A pharmaceutical dosage form according to claim 1 wherein the immediate release component comprises a ratio of carbidopa to levodopa of from about 1:4 to about 1:12.

5. A pharmaceutical dosage form according to claim 1 wherein the in vitro dissolution rate of carbidopa of the immediate release component according to measurements under the USP paddle method of 50 rpm in 900 ml aqueous buffer at pH 4 at 37° C. is from about 50% to about 99% after about 1 hour.

6. A pharmaceutical dosage form according to claim 1 wherein the controlled release component comprises from about 15 to about 85 mg of carbidopa and from 50 to about 500 mg levodopa.

7. A pharmaceutical dosage form according to claim 1 wherein the immediate release component comprises from about 5 to about 50 mg of carbidopa and from 25 to about 300 mg levodopa.

8. A pharmaceutical dosage form having an immediate release component and a controlled release component comprising:
a) an immediate release composition comprising a combination of levodopa and carbidopa in the form of a coating on a solid dosage form;
b) a controlled release composition making up the solid dosage form comprising an active ingredient combination of levodopa and cabidopa together with a retarding agent of a type and amount sufficient to retard the release of active ingredient for a period of from about 1 hour to about 24 hours.
